(19) United States
(12) Patent Application Publication
(10) Pub. No.: US 2019/0316101 A1
(43) Pub. Date: Oct. 17, 2019

(54) METHODS AND COMPOSITIONS FOR GENETICALLY MANIPULATING GENES AND CELLS

(71) Applicant: Howard Hughes Medical Institute, Chevy Chase, MD (US)

(72) Inventors: Tzumin Lee, Chevy Chase, MD (US); Jorge Garcia Marques, Chevy Chase, MD (US); Hui-Min Chen, Chevy Chase, MD (US)

(21) Appl. No.: 16/039,150

(22) Filed: Jul. 18, 2018

Related U.S. Application Data
(60) Provisional application No. 62/533,972, filed on Jul. 18, 2017.

(51) Int. Cl. C12N 9/22; C12N 15/11; C12N 15/63

(52) U.S. Cl. CPC C12N 9/22; C12N 15/111; C12N 15/63; C12N 2310/20; C12N 2510/00; C12N 2330/51; C12N 2800/80

(57) ABSTRACT

This disclosure provides methods and compositions for genetically manipulating genes and cells.

### CROSS REFERENCE TO RELATED APPLICATIONS

This application claims benefit under 35 U.S.C. § 119(e) to U.S. Application No. 62/533,972 filed on Jul. 18, 2017.

### TECHNICAL FIELD

This disclosure generally relates to methods and compositions for genetically manipulating genes and cells.

### BACKGROUND

The discovery of the CRISPR system and its development into a tool for gene editing has undoubtedly sparked one of the major revolutions in research in the last few decades. While most of the developments to date came about through basic research, the precision of the CRISPR system to manipulate specific genes will certainly bring about new applications in areas such as biotechnology and medicine.

### SUMMARY

This disclosure provides compositions and methods that can be used, in conjunction with the CRISPR system, to control gene expression and generate cascades of gene expression, which can be used to boost the computational capacity of the CRISPR system in biological systems.

The methods described herein rely upon the framework of the CRISPR system, particularly regarding the ability of specific RNAs (gRNAs) to direct the activity of a nuclease enzyme (e.g., Cas9 or another nuclease) to modify and control other genetic elements, including reporters, effectors, and even gRNAs themselves.

This disclosure provides methods and compositions that can be used to trigger cascades with specific inputs (e.g., gRNAs). This disclosure also provides methods and compositions that can be used to trigger or inhibit gene expression, or to couple the regulation of a particular gene to the functional state of a certain gRNA. Combining these aspects allows sophisticated biological cascades to be generated with basically unlimited specificities. Thus, the methods described herein allow a user to achieve an unprecedented level of control over the expression of multiple genes across a wide range of time or developmental stages.

In one aspect, a nucleic acid switch cassette is provided that includes a target nucleic acid sequence flanked by first and second direct repeat nucleic acid sequences. In some embodiments, the target nucleic acid sequence in the nucleic acid switch cassette is complementary to a spacer sequence of at least one guideRNA. In some embodiments, the first and second direct repeats are each from about 10 bp in length to about 500 bp in length. In some embodiments, the first and second direct repeats are not identical. In some embodiments, the first and second direct repeats have at least 80% sequence identity (e.g., at least 85%, 90%, 95%, 99% sequence identity) to one another. In some embodiments, the nucleic acid switch cassette is engineered into a desired nucleic acid sequence.

In some embodiments, the switch cassette is an expression-on switch. In some embodiments, the switch cassette is an expression-off switch.

In another aspect, a method is provided that includes engineering a nucleic acid switch cassette into at least one desired nucleic acid sequence, wherein the nucleic acid switch cassette comprises a target nucleic acid sequence flanked by first and second direct repeat nucleic acid sequences; exposing the at least one desired nucleic acid comprising the nucleic acid switch cassette to a CRISPR system, wherein the CRISPR system comprises at least one guide RNA (gRNA) and a nuclease enzyme, wherein a spacer sequence in the at least one gRNA is complementary to the target nucleic acid sequence in the nucleic acid switch cassette, wherein, under appropriate conditions, the nuclease enzyme cleaves the target nucleic acid sequence to which the spacer sequence in the at least one gRNA binds; exposing the cleaved target nucleic acid sequence to single strand annealing (SSA) polypeptides or nucleic acids encoding SSA polypeptides; and determining the on-off status of the nucleic acid switch cassette of the desired nucleic acid sequence based on a phenotype.

In some embodiments, the desired nucleic acid sequence is an open reading frame or a plurality of open reading frames, each comprising a different/unique switch cassette. In some embodiments, the desired nucleic acid sequence is a guide RNA (gRNA) sequence or a plurality of gRNA sequences, each comprising a different/unique switch cassette. In some embodiments, the gRNA sequence is comprised within an open reading frame. In some embodiments, the desired nucleic acid sequence is a promoter sequence.

In some embodiments, the switch cassette is an expression-on switch. In some embodiments, the switch cassette is an expression-off switch.

In still another aspect, a method is provided that includes providing a protein-encoding nucleic acid sequence comprising a nucleic acid switch cassette, wherein the nucleic acid switch cassette comprises a target nucleic acid sequence flanked by first and second direct repeat nucleic acid sequences and disrupts the protein-encoding nucleic acid sequence such that a functional protein is not produced; contacting the disrupted protein-encoding nucleic acid sequence comprising the nucleic acid switch cassette to a CRISPR/Cas9 system, wherein the CRISPR/Cas9 system comprises at least one guide RNA (gRNA) and a Cas9 enzyme, wherein a spacer sequence in the at least one gRNA is complementary to the target nucleic acid sequence in the nucleic acid switch cassette, wherein, under appropriate conditions, the Cas9 enzyme cleaves the target nucleic acid sequence to which the spacer sequence in the at least one gRNA binds; and contacting the cleaved target nucleic acid sequence to single strand annealing (SSA) polypeptides or nucleic acids encoding SSA polypeptides; thereby causing excision of the nucleic acid switch cassette from the disrupted protein-encoding nucleic acid sequence and restoring production of a functional protein.

In another aspect, a method is provided that includes providing a protein-encoding nucleic acid sequence comprising a nucleic acid switch cassette, wherein the nucleic acid switch cassette comprises a target nucleic acid sequence flanked by first and second direct repeat nucleic acid sequences and does not disrupt the protein-encoding nucleic acid sequence such that a functional protein is produced; contacting the protein-encoding nucleic acid sequence comprising the nucleic acid switch cassette to a CRISPR/Cas9 system, wherein the CRISPR/Cas9 system comprises at least one guide RNA (gRNA) and a Cas9 enzyme, wherein a spacer sequence in the at least one gRNA is complementary to the target nucleic acid sequence in the nucleic acid switch cassette, wherein, under appropriate conditions, the Cas9 enzyme cleaves the target nucleic acid sequence to which the spacer sequence in the at least one gRNA binds; and contacting the cleaved target nucleic acid sequence to single strand annealing (SSA) polypeptides or nucleic acids encoding SSA polypeptides; thereby causing excision of the nucleic acid switch cassette from the protein-encoding nucleic acid sequence, which results in disruption of the protein-encoding nucleic acid sequence such that a functional protein is not produced.

In one aspect, a kit is provided that includes at least one cloning vector with direct repeats and a MCS between the direct repeats; at least one gRNA construct designed to accommodate a target sequence within the spacer sequence; and/or at least one CRISPR construct comprising a MCS.

In another aspect, a kit is provided that includes a cloning vector with at least one switch cassette upstream from a MCS; and a plurality of cloning vectors, each comprising a different gRNA, wherein at least one gRNA matches a target sequence. In some embodiments, the kit further includes a MCS for cloning in a promoter of interest in each vector.

In still another aspect, a kit is provided that includes a cloning vector with an ON switch cassette upstream from a MCS; a cloning vector with an OFF switch cassette upstream from a MCS; a cloning vector comprising a nucleic acid sequence encoding a Cas9 enzyme downstream from a MCS; and a cloning vector comprising a first gRNA in the cascade, downstream from a MCS.

In one aspect, a nucleic acid construct is provided that includes, in the 5′ to 3′ direction, a first exon, a disruptor exon and a second exon, wherein the first exon and the second exon together encode a functional moiety, wherein the first splice acceptor sequence comprises a nucleic acid sequence that is complementary to a spacer sequence of a first guide RNA (gRNA).

In some embodiments, the first exon and the second exon together encode a detectable polypeptide. In some embodiments, the first exon and the second exon together encode a second guide RNA. In some embodiments, the cassette is an expression-on cassette. In some embodiments, the cassette is an expression-off cassette.

In another aspect, a method is provided that includes exposing a nucleic acid construct as described herein to a CRISPR system comprising a gRNA and a CRISPR system nuclease.

In still another aspect, a transgenic organism is provided that includes: a first transgenic construct comprising, in the 5′ to 3′ direction, a promoter, at least one guide RNA (gRNA), and a first detectable polypeptide; wherein a germ line of the first transgenic organism comprises a CRISPR system nuclease.

In another aspect, a method is provided that includes: contacting a transgenic organism as described herein with a guide RNA (gRNA); breeding the gRNA-contacted transgenic organism to produce a first progeny having a first phenotype.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

This document contains at least one drawing executed in color. Copies of this patent or patent application publication with color drawings will be provided by the Office upon request and payment of the necessary fee.

FIG. 1 is a schematic of a switch cassette based on SSA repair. Note that the switch cassette is represented like an electrical switch, where the number corresponds to the specific gRNA controlling the activation of that cassette.

FIG. 2A is a schematic in which a switch cassette is inserted in the middle of the GFP open reading frame, thus disrupting it.

FIG. 2B is data from experiments in Drosophila testing the orthogonality of two of these constructs, each with a different gRNA target (gRNA#0 vs. gRNA#1) and a different reporter gene (RFP vs. GFP). The presence of the gRNA #0 (driven by the 44F03 promoter) and Cas9 (driven by the neuroblast-specific DpnEE promoter) results in the reconstitution of the RFP reporter gene (left) but not the GFP reporter gene (right).

FIG. 3-1 and FIG. 3-2 are schematics showing that all the different logic gates for one or two elements (Panel A, “not”; Panel B, “and”; Panel C, “nor”; Panel D, “xor”; Panel E, “nand”; Panel F, “or”; and Panel “xnor”) can be implemented by incorporating one or two switch cassettes (using gRNA #1 and gRNA #2) into the open reading frame of a GFP reporter gene that is active only in phase 0. Numbers between parentheses show the change in phase of the open reading frame, produced after each cassette is activated.

FIG. 4 is a schematic showing the optimization of an activatable gRNA scaffold. Different variants were tested in Drosophila for their ability to trigger a reporter gene in the presence or absence of Cas9 and the gRNA recognizing the specific target in the switch cassette. Variants #3 to #16 exclusively involve the gRNA scaffold and spacer, whereas variant #1 and #2 also contain ribozymes flanking the gRNA to produce the right gRNA only after the activation of the switch cassette. Variant #7 has a switch cassette inserted between the critical sequences (PSEA and TATA box) in the U6 promoter. Some of the variants also have a transcriptional stop signal. Variants #12 and #13 have a random sequence upstream of the gRNA intended to abolish its activity. Variants #5 and #6 exhibited high efficiency cleavage in the presence of Cas9/trigger gRNA and virtually no leakiness in the absence of any of them. This efficiency was tested by crossing flies having one of these constructs to a line with Dpn-Cas9, an U6-gRNA(trigger) and a reporter gene with a target site (similar to that shown in FIG. 2A) for the corresponding spacer (gRNA #2 or #3). Each variant includes a number of full or partial modules. The numbers within each module indicate its length (in bp). Note that some regions have a higher number first (e.g., 23-1), which correspond to inverted sequences. Black lines indicate the homologous regions; orange or green lines indicate the target site. Ribozyme (43 bp), SEQ ID NO:1; gRNA #3 (20 bp), SEQ ID NO:2; gRNA #2 (23 bp), SEQ ID NO:3; gRNA scaffold (20 bp), SEQ ID NO:4; ribozyme 3′ (68 bp), SEQ ID NO:5; target site #2 (23 bp), SEQ ID NO:6; PSEA (27 bp), SEQ ID NO:7; spacer PSEA-TATAbox (9 bp), SEQ ID NO:8; TATA box (8 bp), SEQ ID NO:9; target site #3 (23 bp), SEQ ID NO:10; U6-stop (7 bp), SEQ ID NO:11; random sequence (fragment of neomycin), SEQ ID NO:12.

FIG. 5A shows several regions described in the secondary structure of a normal non-activatable gRNA (Briner et al., 2014, Mol. Cell, 56:333-9; SEQ ID NO:13). Some of these regions (e.g., the Nexus, the Upper Stem) are extremely sensitive to mutations altering their secondary structure.

FIG. 5B shows the sequence of Variant #6 (SEQ ID NO:14), selected for its high efficiency, small size and lack of leaky activity in the absence of Cas9/gRNA. The presence of a target sequence in the middle of the normal gRNA scaffold disrupts its functionality in its non-activated version.

FIG. 5C is an example of a similar activatable gRNA design based on the insertion of a switch cassette in the U6 promoter. This promoter contains two important regulatory sequences, the PSE element and the TATA box. The distance between both elements is critical. The presence of a target site in the middle of both sequences will increase this distance, inhibiting the transcriptional activity of this promoter. Upon the induction of a double-stranded break by Cas9 and the subsequent repair through SSA, both elements will come back to the original separation distance, which will result in the activation of transcription of the downstream gRNA. A solid line shows the extension of the switch cassette (direct repeats in black, target site in orange).

FIG. 6 demonstrates a gRNA cascade in Drosophila. The presence of a specific gRNA (gRNA #1 driven by the 44F03 promoter) simultaneously triggers the RFP reporter gene (left) and a cascade of gRNA in neuroblasts (since Cas9 is expressed under control of the neuroblast specific promoter, DpnEE (“Deadpan”)), which, in turn, triggers expression of the GFP reporter (right). Note the smaller size of green clones, as the gRNA cascade requires multiple steps that delay the activation of the green reporter.

FIG. 7 demonstrates coupling a reporter gene and a gRNA cascade in the HEK293T human cell line. The presence of gRNA #1 induces the activation of gRNA #2 and brings the GFP reporter in-frame (green cells). The activation of gRNA #2 subsequently triggers gRNA #5 and brings the RFP reporter in-frame (red cells; when overlaid with the GFP, results in yellow cells). Some of the cells are only red because they transit to the next step in the cascade, inactivating the GFP and activating the gRNA #4. In the absence of any gRNA or in the presence of a different gRNA (as shown for gRNA #3), virtually none of the cells exhibit fluorescence.

FIG. 8 demonstrates a cascade of reporters based on the coupling between the gRNA activity and the reporter expression. In this case, the cascade is triggered during the development of mushroom bodies in Drosophila. These structures contain three major cell types that are generated in a sequential manner: first the γ(gamma) neurons, which project into the γ(gamma) lobe; second, the α′/β′ (alpha-prime/beta-prime) neurons, projecting to the α′ (alpha-prime) and β′ (beta-prime) lobes; and, finally, the α/β (alpha/beta) neurons, projecting to the α (alpha) and β (beta) lobes. Use of this cascade was able to label the different classes with a different combination of reporters: γ(gamma) neurons in green and green/red; α′/β′ (alpha-prime/beta-prime) neurons in green/red and red; α/β (alpha/beta) neurons only in red.

FIG. 9 illustrates the idea of triggering a cascade of reporters over different generations of flies. For this, Cas9 expression must be transitory in the germ line, but absent in the germ stem cell. Certain promoters such as a Bam promoter can achieve this level of expression. By providing Cas9 and the first gRNA to trigger the cascade, it is possible to induce a single event in the cascade, giving rise to an adult fly with the first reporter activated. This fly will trigger the next step in its germ line, giving rise to the next generation of flies with the second reporter activated, and so on. These methods can be used to activate or deactivate specific transgenes after a certain number of generations.

FIG. 10 shows an embodiment in which a gRNA is used as a mini transcriptional reporter gene. This embodiment takes advantage of the fact that single strand DNA donors are easily integrated after inducing a double strand break into the genome. In this embodiment, the full reporter cassette consists of a very short version of an U6 promoter (with only the critical sequences for transcription), as well as a very compact version of a gRNA (with only those sequences required for a minimal activity). This cassette can be synthesized as a primer (shorter than 200 bp) and used as a donor DNA for gene targeting (with minimal homologous arms). After being inserted into the genome, this gRNA will be active and will trigger the expression of a reporter gene (as shown in FIG. 2A). This will report the success of the integration event.

FIG. 11 illustrates an embodiment to regulate gene expression based on CRISPR/Cas9. In this embodiment, a GFP reporter can be expressed from two different exons. However, a ‘disruptor’ exon abolishes this expression by introducing an additional sequence. This ‘disruptor’ exon can contain a target site for a specific gRNA overlapping with its splicing acceptor. After exposing this construct to CRISPR/Cas9 and a specific gRNA, the splicing acceptor is destroyed. This shifts the splicing to the next exon, thus reconstituting the correct GFP sequence.

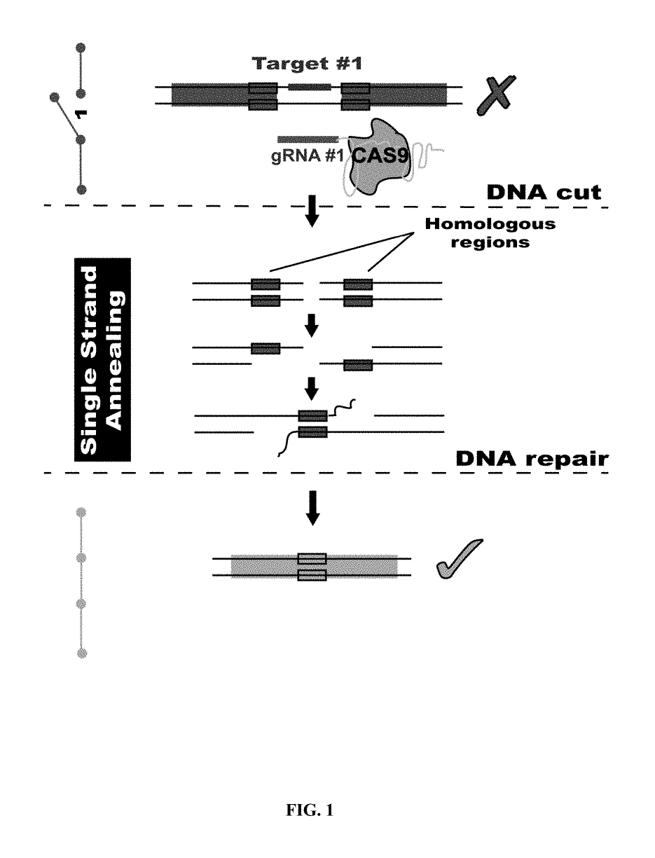

### DETAILED DESCRIPTION

The methods described herein rely on CRISPR as well as an evolutionary-conserved mechanism of DNA repair known as single-strand annealing (SSA). SSA repairs double stranded breaks (DSB) in DNA located between direct repeats by deleting one of the repeats and the intervening sequence (Bhargava et al., 2016, Trends Genet., 23(9):566-75). Since the final outcome of the SSA repair process can be predicted, it is possible to domesticate these mechanisms to obtain a desired outcome. This disclosure takes advantage of this mechanism in the form of a “nucleic acid switch cassette,” which can be used in a variety of constructs and situations.

### The Nucleic Acid Switch Cassette

As used herein, a nucleic acid switch cassette refers to a nucleic acid molecule that includes a target sequence. In some cases the target sequence is flanked by first and second direct repeat sequences. As described in more detail below, a nucleic acid switch cassette can be engineered into a desired nucleic acid sequence. A desired nucleic acid can be virtually any type of genetic element including, without limitation, a coding sequence, a promoter sequence, an enhancer, or a guide RNA (gRNA). For example, a gRNA within a switch cassette can be activated by a second gRNA in the presence of the CRISPR system, which enables gRNA cascades to be constructed (e.g., in cells). In some embodiments, one or more gRNAs can trigger specific genetic responses (e.g., detectable as a phenotype) at one or more steps within a cascade.

Direct repeats involved in SSA are known in the art and can take any number of configurations. For example, either of the direct repeats can be as short as 3 or 5 or 8 or 10 or more nucleotides in length (e.g., 12, 15, 18, 20, 22, 24, 30, 40, 50, 80, 100 or more nucleotides in length), or one or both direct repeats can be as long as, for example, several hundred nucleotides in length (e.g., about 200, 300, 400, 500, 600, 700, 800, 900, 1000 or more nucleotides in length). It would be appreciated that the first and second repeats need not be identical in length or in sequence identity. For example, the first and second direct repeats can have at least 80% sequence identity (e.g., at least 85%, 90%, 95%, 99% sequence identity) to one another over a defined length. It would be understood that it is not the particular sequence or length of sequence in either direct repeat that is important—it is that appropriate sequences are present in each of the first and the second direct repeats to form the secondary structure necessary for SSA.

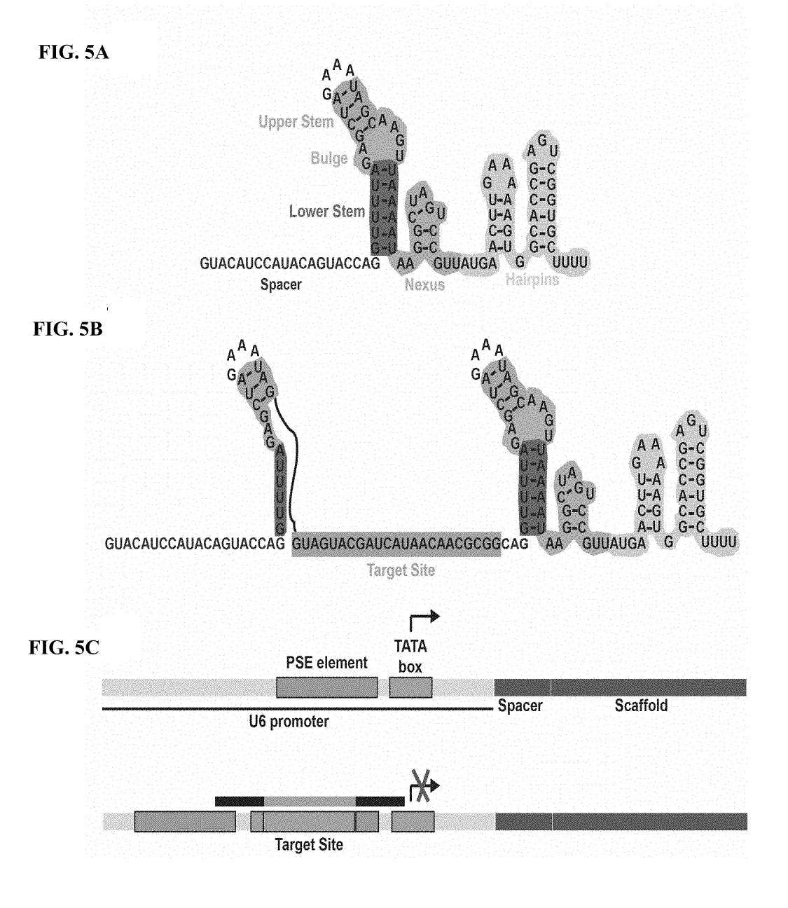

As explained in more detail below, the target nucleic acid sequence within the switch cassette typically is complementary to the spacer sequence of at least one guide RNA (gRNA). gRNAs and their involvement in the CRISPR system are known in the art. Briefly, and as shown in FIG. 5A, the spacer sequence in a gRNA is responsible for the targeting capacity of the CRISPR system, while additional sequences and structures in the gRNA trigger binding and cleavage by the nuclease. See, for example, Briner et al., 2014, Mol. Cell, 56:333-9. As used herein, a gRNA also can refer to the wild type form (crRNA+tracrRNA).

Nucleic acids as used herein can include DNA and RNA, and also includes nucleic acids that contain one or more nucleotide analogs. A nucleic acid can be single-stranded or double-stranded, which will depend upon its intended use.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

Changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

Nucleic acid fragments also are included. Nucleic acid fragments suitable for use in the methods described herein are fragments that encode a polypeptide having functional activity. These fragments can be referred to as “functional fragments,” although it is understood that it is not the nucleic acid that possesses functionality.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A construct or vector containing a nucleic acid (e.g., a nucleic acid that encodes a polypeptide) also is provided. Constructs or vectors, including expression vectors, are commercially available or can be produced by recombinant DNA techniques routine in the art. A construct or vector containing a nucleic acid can have expression elements operably linked to such a nucleic acid, and further can include sequences such as those encoding a selectable marker (e.g., an antibiotic resistance gene). A construct or vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid (e.g., in-frame). Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Constructs and vectors as described herein can be introduced into a host cell. As used herein, “host cell” refers to the particular cell into which the nucleic acid is introduced and also includes the progeny or potential progeny of such a cell. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are pre-hybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, Calif.).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

### Methods of Using a Switch Cassette

There are numerous ways in which a switch cassette as described herein can be used. The following descriptions are only intended to be representative and are not intended to be exhaustive.

The initial step of the methods described herein includes engineering a nucleic acid switch cassette into at least one desired nucleic acid sequence. As used herein, a desired nucleic acid sequence can be an open reading frame, a guide RNA (gRNA) sequence, a promoter sequence, an enhancer sequence or any number of other types of genetic elements. In one embodiment, the desired nucleic acid sequence is an open reading frame that contains a gRNA sequence within. It would be appreciated that the desired nucleic acid sequence can be a plurality of desired nucleic acid sequences (e.g., a plurality of open reading frames, each comprising a different or unique switch cassette or a plurality of gRNA sequences, each comprising a different or unique switch cassette).

The next step of the methods described herein includes exposing the desired nucleic acid containing the nucleic acid switch cassette to a CRISPR system. CRISPR systems are known in the art and includes at least one guide RNA (gRNA) and a nuclease enzyme (e.g., one or more nucleic acids encoding a gRNA and a nuclease enzyme). See, for example, U.S. Pat. Nos. 8,697,359; 8,889,418; 8,999,641; and US 2014/0068797. In the present methods, the gRNA (i.e., the spacer sequence in the gRNA) is fully or partially complementary to the target nucleic acid sequence in the switch cassette, such that, under appropriate conditions, the nuclease enzyme cleaves the target nucleic acid sequence to which the gRNA (i.e., the spacer sequence in the gRNA) binds.

The next step of the methods described herein includes exposing the cleaved switch cassette (i.e., the cleaved target nucleic acid sequence) to single strand annealing (SSA) polypeptides (e.g., one or more nucleic acids encoding such SSA polypeptides). In the presence of the SSA polypeptides, the cleaved switch cassette is precisely excised from the desired nucleic acid sequence, whereas any uncleaved switch cassette (i.e., a switch cassette having a target sequence that is not complementary to a gRNA in the CRISPR system) are not excised and are retained within the desired nucleic acid sequence. It would be appreciated that a phenotype (e.g., due to excision of the switch cassette from the desired nucleic acid or due to the switch cassette not being excised from the desired nucleic acid) can be observed after the initial, or first, excision (or lack of excision). Alternatively, it would be appreciated that a phenotype can be observed after multiple excisions (or lack of excisions), or that one or more phenotypes can be observed after multiple excisions (or lack of excisions). The multitude of excision or non-excision events and pathways that can be generated with the compositions and methods described herein (the “cascade”) creates essentially an infinite number of potential uses and applications.

It would be understood that a switch cassette can be an “on” switch or an “off” switch. For example, in an on-switch cassette, the desired nucleic acid sequence can be a protein-encoding nucleic acid sequence such that, in the presence of the switch cassette, the coding sequence is disrupted and a functional protein is not produced. Following cleavage of the target sequence by the CRISPR system and repair by SSA to excise the switch cassette, however, the coding sequence is restored and a functional protein is produced. Similarly, in a switch-on cassette, the desired nucleic acid sequence can be a promoter such that, in the presence of the switch cassette, the promoter is disrupted and is not able to drive expression of a coding sequence. As described herein, following cleavage of the target sequence by the CRISPR system and subsequent repair by SSA to excise the switch cassette, the promoter is restored and able to drive expression of a coding sequence.

It would be understood that, in an off-switch cassette, the desired nucleic acid sequence also can be a coding sequence or a promoter sequence, but that this configuration would require more careful engineering of the switch cassette such that, when present, expression of the coding sequence or activity of the promoter sequence is not disrupted. In an off-switch configuration, the expression of the coding sequence or the activity of the promoter sequence is disrupted when the switch cassette is excised (following action by the CRISPR system and the SSA polypeptides), thereby resulting in the loss of a protein or loss of a functional protein.

It would be understood that the desired nucleic acid containing the switch cassette is contacted with the CRISPR system and with the SSA polypeptides under appropriate conditions. Although the switch cassettes and the associated methods described herein can be used in vitro, it is envisioned that their value lies in the ability to use them, individually or in combinations, in vivo. As used herein, in vivo refers to in cells or tissues, whether those cells or tissues are in culture or reside within a higher organism (e.g., a rodent, a fish, a plant, a primate, a human, or any other appropriate eukaryotic or prokaryotic cell). It would be understood, therefore, that the components of the CRISPR system and the SSA polypeptides can be provided to the cells exogenously (e.g., as proteins (e.g., SSA polypeptides and a CRISPR-appropriate nuclease) and functional nucleic acids (e.g., gRNA for the CRISPR system)), or the components of either or both the CRISPR system and the SSA polypeptides can exist endogenously or be genetically engineered to be expressed in the cell.

Cascades that employ switch cassettes and gRNAs as described herein can be used to control cell specification, which can be useful when inducing pluripotent stem cells for therapeutic purposes or for drug screening.

Additionally or alternatively, once activated, the gRNAs can be detected, for example, using in situ hybridization. This is an alternative way to identify a gRNA cascade, rather than using the gRNAs to trigger the expression of reporter genes.

Similarly, cascades that employ switch cassettes and gRNAs as described herein can be applied to metabolic engineering. For example, the production of a drug or biologic frequently involves the introduction of several genes of a biosynthetic pathway into a heterologous host. Efficient production of the drug or biologic, however, requires a precise balance of expression of the genes (e.g., so that no gene is drastically overexpressed, which would deplete precursors for growth and product biosynthesis, or drastically under-expressed, which would create a bottleneck in the metabolic pathway and limit product synthesis). The switch cassettes and methods described herein can be used to precisely control, for example, the timing and/or cellular location for expression of one of more of the required genes.

Alternatively, these cascades can be used as a genetic memory (e.g., a recording of how long a biological system has been exposed to a certain factor). For example, the longer the exposure, the more progression is observed in the cascade. This type of cascade would allow sensing to determine how long a certain cell has been exposed to one or more factors. For example, a different response (e.g., phenotype) can be triggered for each exposure or combination thereof (e.g., factor A, factor B, or both factors A & B).

Since the specificity of nucleases involved in CRISPR systems such as Cas9 and other Cas variants are virtually unlimited, the applications of the switch cassettes and the cascades utilizing them also are virtually unlimited. Initially, this system was used to create a cascade of different reporter genes, which can allow, for example, cell lineages to be traced. A further layer of complexity can be provided by expressing the CRISPR-associated nuclease (e.g., Cas9 or another Cas variant) under different promoters. Such promoters can include, for example, promoters that are dependent upon transcription factor activity, neural activity, circadian rhythms, etc. In addition, switch cassettes as described herein can be used to generate multicolor reporters capable of labeling a very small number of cells having a specific combination of epitopes among thousands of cells.

Various combinations of switch cassettes and gRNAs can be used, for example, to allow expression of a specific gene only in the presence of a specific combination of promoters. One configuration, for example, will allow expression of a given gene only in a particular sub-population of cells expressing a specific combination of markers. Also as described herein, various combinations of promoters also can be used to control where and when various steps of the cascade take place. For example, the nuclease enzyme can be under control of a developmental-specific or tissue-specific promoter such that one or more switch cassettes are contacted with the CRISPR system (e.g., a viable CRISPR system) only in particular cells or tissues or only at particular developmental stages. For example, if the nuclease enzyme is under control of a promoter that is only active during mitosis, then the cascade (e.g., expression of a gene, or disruption of the expression of a gene) only would occur during mitosis of the cells.

There currently is not a similar system that can be used to achieve genetic intersection with an unlimited number of elements.

### Articles of Manufacture or Kits

This disclosure also provides for articles of manufacture (or kits) containing one or more components as described herein. These articles of manufacture or the components therein can be used to carry out any number of methods, whether described or exemplified herein or not. For example, a kit as described herein can include, without limitation, one or more cloning vectors having direct repeats and a multiple cloning site (MCS) therebetween for cloning in the target nucleic acid sequence; one or more gRNA constructs designed to accommodate a desired spacer sequence; and/or a construct encoding the CRISPR system components and having a MCS for cloning in the spacer sequence.

In one embodiment, a kit is provided with components as described herein to achieve genetic intersection (e.g., logic gates). Such a kit can include, for example, a cloning vector with at least one switch cassette upstream of a MCS (e.g., for cloning in an open reading frame); and a plurality of cloning vectors, each having a different gRNA, wherein at least one gRNA matches (e.g., is complementary to) the target sequence in the at least one switch cassettes. In some instances, the plurality of cloning vectors can further include an upstream MCS (e.g., for cloning in a promoter of interest).

In one embodiment, a kit is provided with components as described herein to trigger genetic cascades. Such a kit can include, for example, a cloning vector with an ON switch cassette upstream from a MCS (e.g., for cloning in a desired open reading frame or in a promoter); and/or a cloning vector with an OFF switch cassette upstream from a MCS (e.g., for cloning in a desired open reading frame or in a promoter). For example, the activation of the ON switch cassette will trigger the open reading frame and produce a gRNA that, in turn, will trigger the next step in the cascade; the activation of the OFF switch cassette will stop the open reading frame expression and will trigger the next step in this cascade. Such a kit also can include a cloning vector that includes a nucleic acid sequence encoding a CRISPR-associated nuclease enzyme downstream from a MCS (e.g., for cloning in the desired promoter); and/or a cloning vector that includes a gRNA (e.g., the first gRNA in the cascade) downstream from a MCS (e.g., for cloning in a promoter of interest).

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Controlling Gene Expression with gRNAs

Some embodiments described herein include one or more switch cassettes within an open reading frame (ORF), where the final repaired outcome is an activated (in-frame) or inactivated (out-of-frame) form of a gene. In these cases, the repeat length can be as short as 10 bp or as long as 500 bp, and there can be a certain number of the same or different target sites.

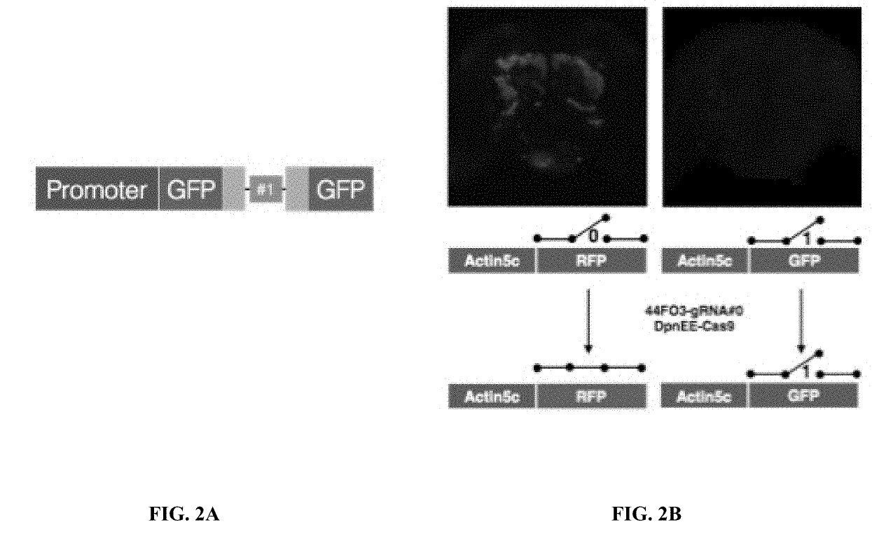

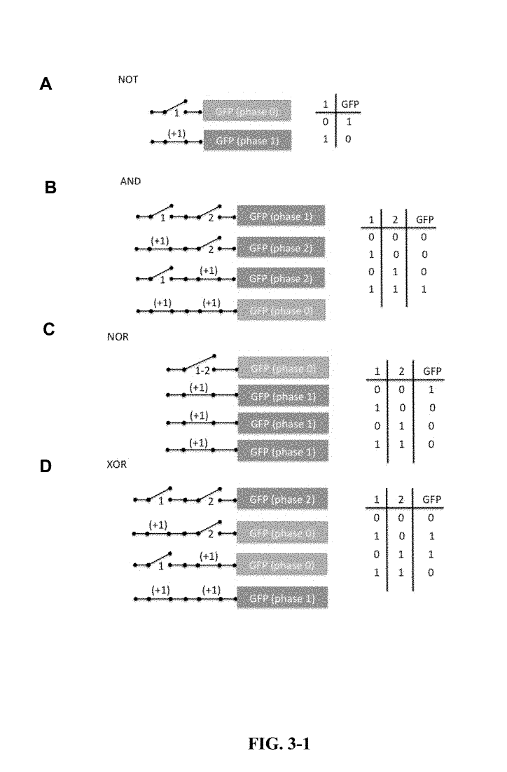

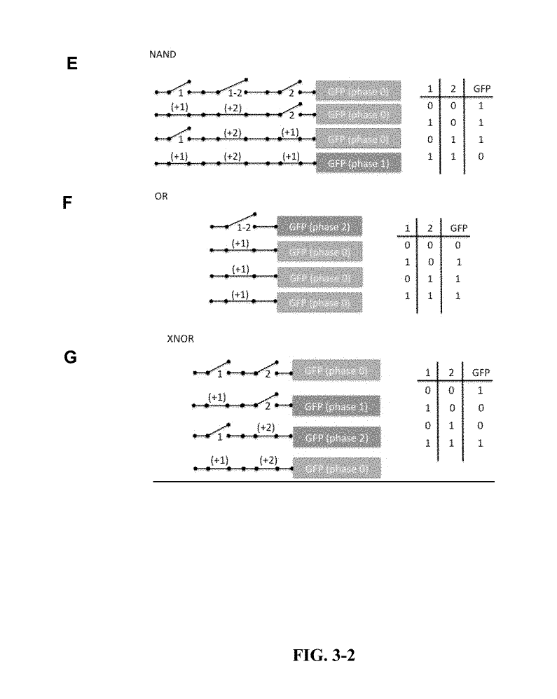

In one embodiment, the presence of a specific gRNA activates a specific gene, as was demonstrated in experiments using a reporter gene in Drosophila (FIG. 2). In another embodiment, a similar design can be implemented using two or more different switch cassettes to allow implementation of all the basic logic gates (FIG. 3).

### Example 2—A gRNA Scaffold Activated by gRNA

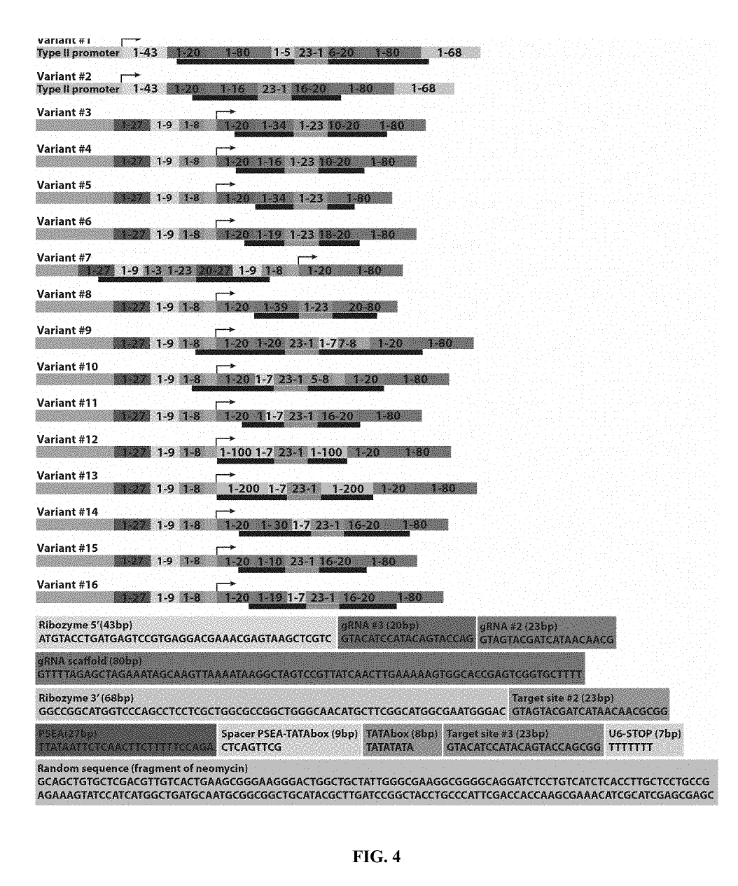

In some embodiments, a switch cassette can be inserted into a gRNA, thereby inactivating it. Once Cas9 induces a double-stranded break in the specific target site in the cassette, a reconstituted active gRNA is formed. To optimize this design and avoid leakiness in the inactive form, multiple alternative gRNA scaffolds were generated (FIG. 4). Two different versions were obtained with no leakiness observed in the absence of either Cas9 or the trigger gRNA; both versions exhibited similar efficiency in their ability to be reconstituted by SSA and then trigger a reporter gene (similar to that shown in FIG. 2). These variants, gRNAs #5 and #6, have direct repeats of 34 and 22 bp, respectively. gRNA variant #6 was chosen for further experiments due simply to its shorter length.

The first part of the variant #6 sequence contains the spacer and most of the first hairpin, including the lower stem, upper stem and bulge (FIG. 5). Downstream of the target site, the second repeat covers 3 bp of spacer and the rest of the scaffold. It was expected that any variant not including the entire nexus in the first part and less than 7 bp of spacer in the second part would not exhibit leakiness.

In some embodiments, a switch cassette can be integrated within a promoter (e.g., a U6 or similar promoter such as, but not limited to, a H1 promoter). For example, integrating a switch cassette as described herein between a proximal sequence element (PSE) and a TATA box disrupts transcription of the gRNA (since the spacing between is critical; see, for example, Ventura et al., 2004, PNAS USA, 101(28):10380-5). After action by a Cas9 enzyme, both elements come together, enabling transcription from that promoter.

### Example 3—gRNA Cascades

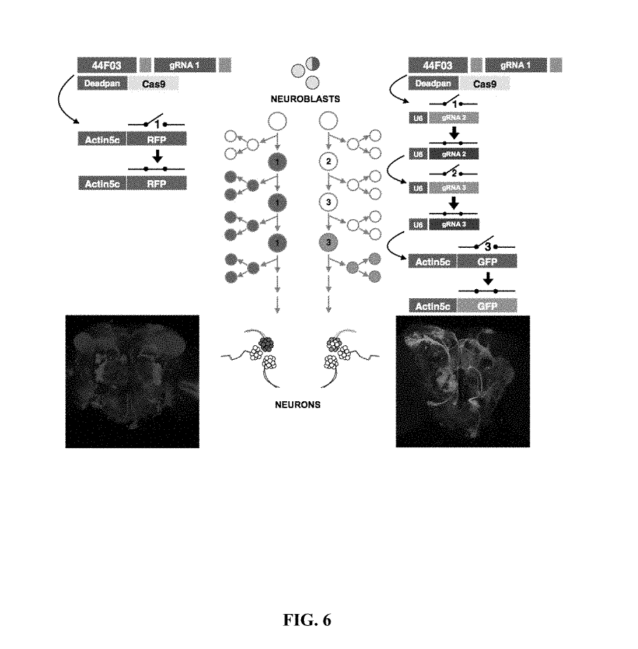

By taking advantage of the above-described designs, this disclosure also provides different compositions that can be used to trigger cascades of an unlimited number of gRNAs. For example, a certain gRNA will trigger the subsequent activation of another, and so on and so forth in a cascade of gRNA activation. Each of these gRNAs then may be used to activate or deactivate different genes and/or other gRNA cascades, and/or they may perform any of the functions of a regular gRNA (e.g., gene editing). The feasibility of these cascades was demonstrated in Drosophila (FIG. 6).

### Example 4—Coupling gRNA Activation to Gene Expression

Even when each gRNA in the cascade controls the activation/deactivation of specific genes, this will not necessarily occur in a synchronous way. In some instances, for example, a gRNA will be activated without the concurrent activation/deactivation of the gene. Thus, the gRNA cascade will progress faster or slower than the gene activation/deactivation cascade.

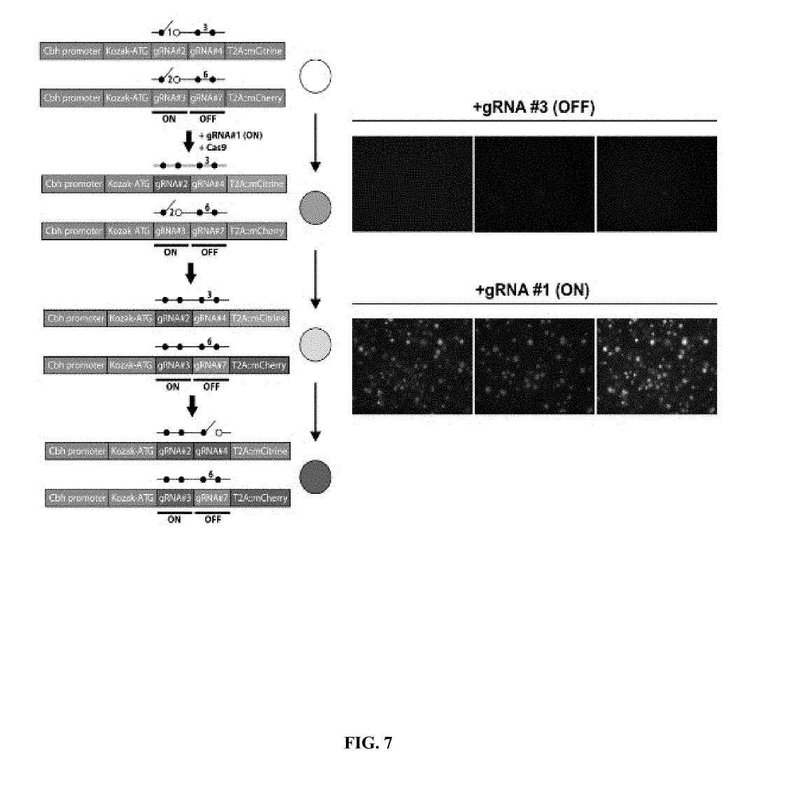

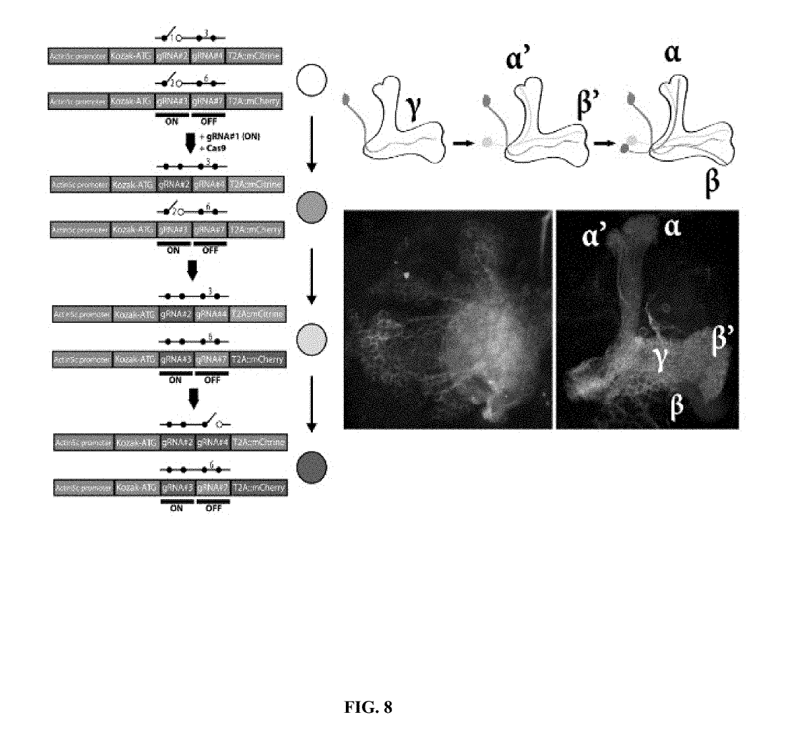

This disclosure provides a design to couple a certain gRNA to a gene by introducing an activatable gRNA within the open reading frame of that gene. In this way, only when the gRNA is activated will that gene be in frame (or out of frame, in the case of deactivation). Thus, the functional state of a gRNA will directly determine the expression of the gene. A similar design can incorporate two different activatable gRNAs so as to couple each of them to the activation or deactivation of the gene. This embodiment has been demonstrated in the human cell line HEK297T (FIG. 7) as well as in Drosophila (FIG. 8).

### Example 5—Multi-Generation Cascades

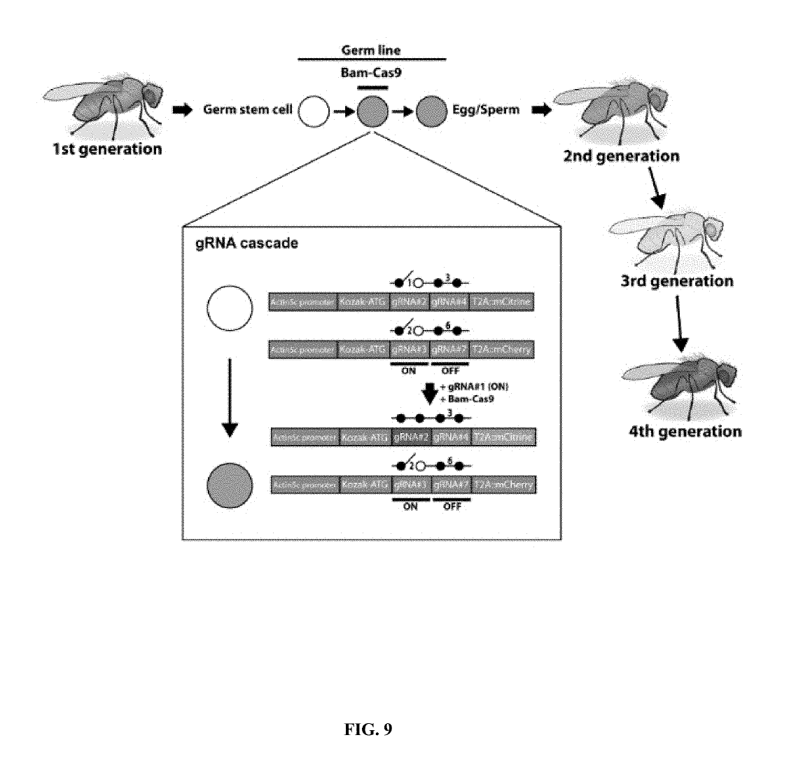

FIG. 9 shows an embodiment in which a cascade of gRNAs can be triggered over different generations (e.g., of flies or another organism). To do this, Cas9 or another nuclease involved in a CRISPR system is expressed in the germ line, but not in the germ stem cell, of a fly or another organism. This expression must be transitory, so that the cascade will only progress one or few steps for each generation. A transitory promoter such as a Bam promoter from Drosophila can be used (Chen and McKearin, Development, 2003). This promoter comprises three parts: a) an germ cell specific enhancer (AAA TTG AAA CTT ATT TGT GCG ACG GC (SEQ ID NO:15)); b) a region that can be replaced by basal promoter elements (GTA CTC GAC ATG ATA TCG ATA CGT TAA (SEQ ID NO:16)); c) a silencer CGC AGA CAG CGT GGC GTC (SEQ ID NO:17)) to repress the expression in the germ stem cell. Similar regulatory regions from ortholog genes in other organisms can also be used (e.g., GM114, Tang et al., 2008, Dev. Biol., 318:73-81). Such an embodiment can be used in a technology called Gene Drives, which could help in pest control (see, for example, harvard.edu/media-post/crispr-cas9-gene-drives/ on the World Wide Web). The embodiment described herein can trigger specific genes and/or gRNAs after a certain number of generations. This could be also used to regulate how a gene drive spreads in a population, by triggering or suppressing different gene drivers after a certain number of generations.

### Example 6—Shorter Transcriptional Reporter Genes

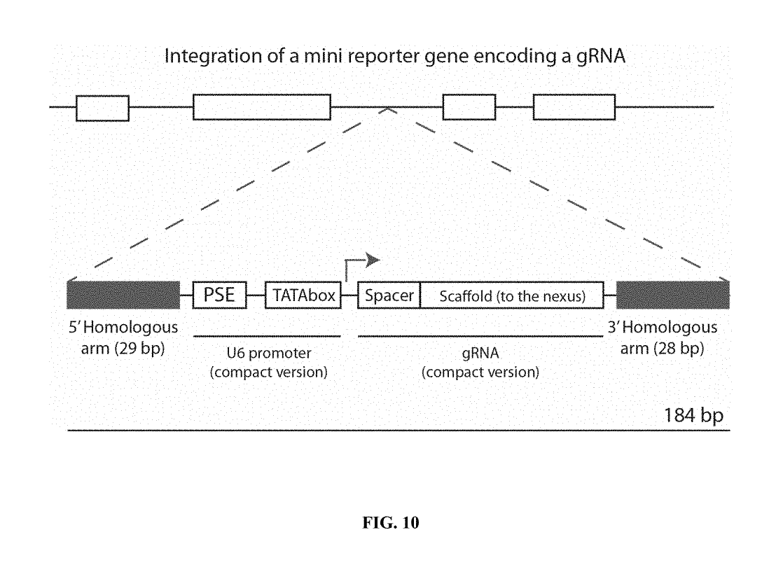

In another embodiment, this technology can be used to create a very short transcriptional reporter gene (FIG. 10). To do that, a very short promoter or a reduced version of any other promoter (such as the U6 promoter) with only the required regulatory sequences (e.g., proximal sequence element and TATA box; Jensen et al., 1998, Nucleic Acids Res., 26:616-22) can be used, which will drive a short version of a gRNA, again, with only the critical sequences (from the spacer to the nexus; Briner et al., 2014, Mol. Cell, 56:333-9). In the example with the U6 promoter, this expression unit is shorter than 200 bp in length. Alternatively, this reporter only will consist of a short promoter and a crRNA. By expressing this small construct, a protein such as a reporter gene (e.g., GFP) can be triggered (as shown in FIG. 2A).

The small size of this construct makes it very useful. For instance, gene targeting using primers as a donor instead of long DNAs is very efficient, but requires a very tedious screening process because a typical primer cannot contain a reporter gene. The small expression construct described herein, however, solves this problem. Moreover, as this reporter only requires transcription, it also can be inserted in regions other than open reading frames such as introns. This will not interrupt the expression of the targeted gene, preventing deleterious effects in the cells. In another embodiment, the donor DNA for this mini reporter is assembled by annealing two primers (with an homologous region) either in vitro (e.g., by increasing the temperature over 95° C. and then decreasing it progressively) or in vivo (e.g., by transfecting/transforming both primers in the same cell).

### Example 7—an Alternative Way to Achieve a Similar Control of Gene Expression

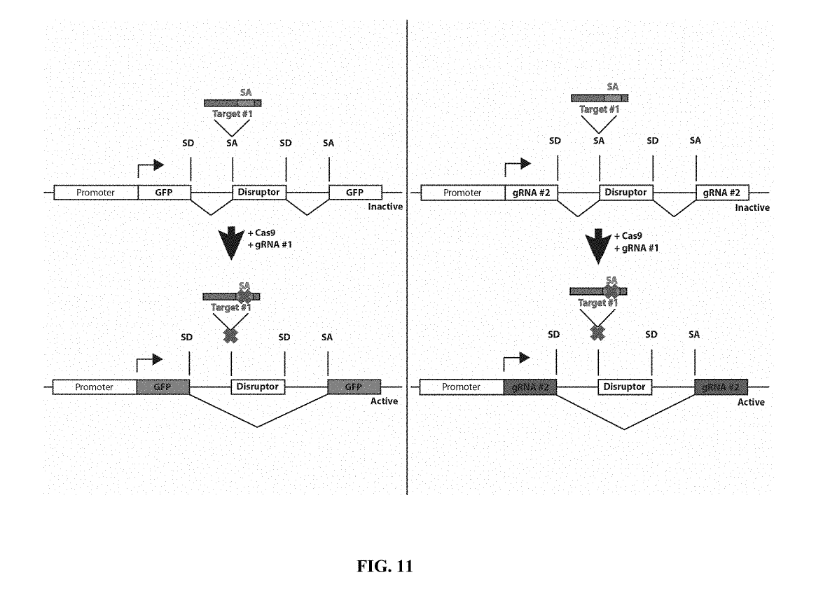

FIG. 11 shows a schematic in which an exon is used to disrupt an open reading frame or a gRNA. The exon contains a target site for a specific gRNA in the splicing acceptor. Alternatively, a cassette with the splicing acceptor located between two direct repeats can be used. After a double strand break produced by Cas9, the splicing acceptor is destroyed (either by non-homologous end joining or SSA) and the next exon will be used. This can either restore or disrupt the open reading frame or the gRNA, based on different designs. Splicing acceptor sequences are known in the art and, given a splicing acceptor sequence, a skilled artisan would understand where the target (e.g., a gRNA) should be positioned.

For control of the gRNA state, this system requires the gRNA to be expressed from type II promoters. Thus, ribozymes or trRNA can be included that flank the gRNA. These features are known in the art and are used routinely.

### Example 8—A Second Alternative Way to Achieve a Similar Control of Gene Expression

In this case, a variant of a CRISPR-associated nuclease induces editing in a specific nucleotide (such as, but not limited to, nickaseCas9-cytidine deaminase, nickaseCas9-APOBEC and other similar editors known in the field). In some embodiments, this edition event will modify a starting codon or generate a stop codon, therefore suppressing the expression of a protein. In other embodiments, the edition event will produce a starting codon or destroy a stop codon, activating the expression of a protein. In other designs, the edition event will activate or inactivate the function of a gRNA by modifying the sequence in the spacer or in the target that this gRNA has to recognize. In another embodiment, the edition event will happen in the scaffold of this gRNA so the secondary structure will be modified, making this gRNA either functional or inactive. In different designs, this edition event takes place in the regulatory regions controlling the expression of a protein or a gRNA. This will suppress or activate the protein or gRNA. This event could also happen in the 5′UTR of a mRNA so this leads to suppress or activate its translation. In other embodiments, this event will destroy or create a new splicing donor or acceptor. This will change the order of the splicing, therefore, changing the final sequence of a protein or gRNA and activating or suppressing its activity (as explained in Example 7).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A nucleic acid switch cassette comprising a target nucleic acid sequence flanked by first and second direct repeat nucleic acid sequences.

2. The switch cassette of claim 1, wherein the target nucleic acid sequence in the nucleic acid switch cassette is complementary to a spacer sequence of at least one guideRNA.

3. The switch cassette of claim 1, wherein the first and second direct repeats are each from about 3 bp in length to about 1000 bp in length.

4. The switch cassette of claim 1, wherein the first and second direct repeats are not identical.

5. The switch cassette of claim 1, wherein the first and second direct repeats have at least 80% sequence identity, at least 85% sequence identity, at least 90% sequence identity, at least 95% sequence identity, or at least 99% sequence identity to one another.

6. The switch cassette of claim 1, wherein the nucleic acid switch cassette is engineered into a desired nucleic acid sequence.

7. The switch cassette of claim 1, wherein the switch cassette is an expression-on switch.

8. The switch cassette of claim 1, wherein the switch cassette is an expression-off switch.

9. A method, comprising
engineering a nucleic acid switch cassette into at least one desired nucleic acid sequence, wherein the nucleic acid switch cassette comprises a target nucleic acid sequence flanked by first and second direct repeat nucleic acid sequences;
exposing the at least one desired nucleic acid comprising the nucleic acid switch cassette to a CRISPR system, wherein the CRISPR system comprises at least one guide RNA (gRNA) and a nuclease enzyme, wherein a spacer sequence in the at least one gRNA is complementary to the target nucleic acid sequence in the nucleic acid switch cassette, wherein, under appropriate conditions, the nuclease enzyme cleaves the target nucleic acid sequence to which the spacer sequence in the at least one gRNA binds;
exposing the cleaved target nucleic acid sequence to single strand annealing (SSA) polypeptides or nucleic acids encoding SSA polypeptides; and
determining the on-off status of the nucleic acid switch cassette of the desired nucleic acid sequence based on a phenotype.

10. The method of claim 9, wherein the desired nucleic acid sequence is an open reading frame.

11. The method of claim 9, wherein the desired nucleic acid sequence comprises a plurality of open reading frames, each comprising a different switch cassette.

12. The method of claim 9, wherein the desired nucleic acid sequence is a guide RNA (gRNA) sequence.

13. The method of claim 12, wherein the gRNA sequence is comprised within an open reading frame.

14. The method of claim 9, wherein the desired nucleic acid sequence comprises a plurality of gRNA sequences, each comprising a different switch cassette.

15. The method of claim 9, wherein the desired nucleic acid sequence is a promoter sequence.

16. The method of claim 9, wherein the switch cassette is an expression-on switch.

17. The method of claim 9, wherein the switch cassette is an expression-off switch.

18. A method, comprising
providing a protein-encoding nucleic acid sequence comprising a nucleic acid switch cassette, wherein the nucleic acid switch cassette comprises a target nucleic acid sequence flanked by first and second direct repeat nucleic acid sequences and disrupts the protein-encoding nucleic acid sequence such that a functional protein is not produced;
contacting the disrupted protein-encoding nucleic acid sequence comprising the nucleic acid switch cassette to a CRISPR/Cas9 system, wherein the CRISPR/Cas9 system comprises at least one guide RNA (gRNA) and a Cas9 enzyme, wherein a spacer sequence in the at least one gRNA is complementary to the target nucleic acid sequence in the nucleic acid switch cassette, wherein, under appropriate conditions, the Cas9 enzyme cleaves the target nucleic acid sequence to which the spacer sequence in the at least one gRNA binds; and
contacting the cleaved target nucleic acid sequence to single strand annealing (SSA) polypeptides or nucleic acids encoding SSA polypeptides;
thereby causing excision of the nucleic acid switch cassette from the disrupted protein-encoding nucleic acid sequence and restoring production of a functional protein.

19. A method, comprising
providing a protein-encoding nucleic acid sequence comprising a nucleic acid switch cassette, wherein the nucleic acid switch cassette comprises a target nucleic acid sequence flanked by first and second direct repeat nucleic acid sequences and does not disrupt the protein-encoding nucleic acid sequence such that a functional protein is produced;
contacting the protein-encoding nucleic acid sequence comprising the nucleic acid switch cassette to a CRISPR/Cas9 system, wherein the CRISPR/Cas9 system comprises at least one guide RNA (gRNA) and a Cas9 enzyme, wherein a spacer sequence in the at least one gRNA is complementary to the target nucleic acid sequence in the nucleic acid switch cassette, wherein, under appropriate conditions, the Cas9 enzyme cleaves the target nucleic acid sequence to which the spacer sequence in the at least one gRNA binds; and
contacting the cleaved target nucleic acid sequence to single strand annealing (SSA) polypeptides or nucleic acids encoding SSA polypeptides;
thereby causing excision of the nucleic acid switch cassette from the protein-encoding nucleic acid sequence, which results in disruption of the protein-encoding nucleic acid sequence such that a functional protein is not produced.

20. A kit, comprising:
at least one cloning vector with direct repeats and a MCS between the direct repeats;
at least one gRNA construct designed to accommodate a target sequence within the spacer sequence; and/or
at least one CRISPR construct comprising a MCS.

21. A kit, comprising:
a cloning vector with at least one switch cassette upstream from a MCS; and
a plurality of cloning vectors, each comprising a different gRNA, wherein at least one gRNA matches a target sequence.

22. The kit of claim 21, further comprising a MCS for cloning in a promoter of interest in each vector.

23. A kit, comprising:
a cloning vector with an ON switch cassette upstream from a MCS;
a cloning vector with an OFF switch cassette upstream from a MCS;
a cloning vector comprising a nucleic acid sequence encoding a Cas9 enzyme downstream from a MCS; and
a cloning vector comprising a first gRNA in the cascade, downstream from a MCS.

24. A nucleic acid construct comprising, in the 5′ to 3′ direction, a first exon, a disruptor exon and a second exon, wherein the first exon and the second exon together encode a functional moiety, wherein the first splice acceptor sequence comprises a nucleic acid sequence that is complementary to a spacer sequence of a first guide RNA (gRNA).

25. The nucleic acid construct of claim 24, wherein the first exon and the second exon together encode a detectable polypeptide.

26. The nucleic acid construct of claim 24, wherein the first exon and the second exon together encode a second guide RNA.

27. The nucleic acid construct of claim 24, wherein the cassette is an expression-on cassette.

28. The nucleic acid construct of claim 24, wherein the cassette is an expression-off cassette.

29. A method comprising:
exposing the nucleic acid construct of claim 24 to a CRISPR system comprising a gRNA and a CRISPR system nuclease.

30. A transgenic organism comprising:
a first transgenic construct comprising, in the 5′ to 3′ direction, a promoter, at least one guide RNA (gRNA), and a first detectable polypeptide;
wherein a germ line of the first transgenic organism comprises a CRISPR system nuclease.

31. A method, comprising:
contacting the transgenic organism of claim 30 with a guide RNA (gRNA);
breeding the gRNA-contacted transgenic organism to produce a first progeny having a first phenotype.


SEQUENCE LISTING (17 sequences)

SEQ ID NO: 1 - DNA, 43 bp, Artificial
  synthetic oligonucleotide
atgtacctgatgagtccgtgaggacgaaacgagtaagctcgtc

SEQ ID NO: 2 - DNA, 20 bp, Artificial
  synthetic oligonucleotide
gtacatccatacagtaccag

SEQ ID NO: 3 - DNA, 20 bp, Artificial
  synthetic oligonucleotide
gtagtacgatcataacaacg

SEQ ID NO: 4 - DNA, 80 bp, Artificial
  synthetic oligonucleotide
gttttagagctagaaatagcaagttaaaataaggctagtccgttatcaacttgaaaaagtggcaccgagtcggtgctttt

SEQ ID NO: 5 - DNA, 68 bp, Artificial
  synthetic oligonucleotide
ggccggcatggtcccagcctcctcgctggcgccggctgggcaacatgcttcggcatggcgaatgggac

SEQ ID NO: 6 - DNA, 26 bp, Artificial
  synthetic oligonucleotide
gtagtacgatcataacaacaacgcgg

SEQ ID NO: 7 - DNA, 27 bp, Artificial
  synthetic oligonucleotide
ttataattctcaacttctttttccaga

SEQ ID NO: 8 - DNA, 9 bp, Artificial
  synthetic oligonucleotide
ctcagttcg

SEQ ID NO: 9 - DNA, 8 bp, Artificial
  synthetic oligonucleotide
tatatata

SEQ ID NO: 10 - DNA, 23 bp, Artificial
  synthetic oligonucleotide
gtacatccatacagtaccagcgg

SEQ ID NO: 11 - DNA, 7 bp, Artificial
  synthetic oligonucleotide
ttttttt

SEQ ID NO: 12 - DNA, 198 bp, Artificial
  synthetic oligonucleotide
gcagctgtgctcgacgttgtcactgaagcgggaagggactggctgctattgggcgaaggcggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagc

SEQ ID NO: 13 - RNA, 99 nt, Artificial
  synthetic oligonucleotide
guacauccauacaguaccagguuuugagcuagaaauagcaaguuaaaauaaggcuaguccguuaugaacuugaaaaaguggcaccgagucggugcuuuu

SEQ ID NO: 14 - RNA, 145 nt, Artificial
  synthetic oligonucleotide
guacauccauacaguaccagguuuuagagcuagaaauagguaguacgaucauaacaacgcggcagguuuuagagcuagaaauagcaaguuaaaauaaggcuaguccguuaugaacuugaaaaaguggcaccgagucggugcuuuu

SEQ ID NO: 15 - DNA, 26 bp, Artificial
  synthetic oligonucleotide
aaattgaaacttatttgtgcgacggc

SEQ ID NO: 16 - DNA, 27 bp, Artificial
  synthetic oligonucleotide
gtactcgacatgatatcgatacgttaa

SEQ ID NO: 17 - DNA, 18 bp, Artificial
  synthetic oligonucleotide
cgcagacagcgtggcgtc